(19) United States
(12) Patent Application Publication
(10) Pub. No.: US 2016/0325022 A1
(43) Pub. Date: Nov. 10, 2016

(54) COMPOSITE SYNTHETIC NANOFIBROUS SCAFFOLDS AND ARTICLES THEREOF

(71) Applicant: 3M INNOVATIVE PROPERTIES COMPANY, Saint Paul, MN (US)

(72) Inventors: Jie Liu, Cottage Grove, MN (US); Amy K. McNulty, Stillwater, MN (US)

(21) Appl. No.: 15/109,381

(22) PCT Filed: Dec. 22, 2014
(86) PCT No.: PCT/US2014/071787

§ 371 Date: Jun. 30, 2016

Related U.S. Application Data
(60) Provisional application No. 61/922,617, filed on Dec. 31, 2013.

(51) Int. Cl. A61L 27/60; A61L 27/54; A61L 27/26; A61L 27/24; D01D 1/02; A61L 27/22; A61L 27/38; A61L 27/36; D01D 5/00; A61L 27/58; A61L 27/20

(52) U.S. Cl. CPC A61L 27/60; A61L 27/58; A61L 27/54; A61L 27/26; A61L 27/24; A61L 27/20; A61L 27/222; A61L 27/3839; A61L 27/3804; A61L 27/3813; A61L 27/3886; A61L 27/362; D01D 5/003; D01D 1/02; A61L 2300/42; A61L 2430/34; A61L 2300/604; A61L 2400/12; D10B 2509/00

(57) ABSTRACT

A synthetic scaffold that comprises a population of nanofibers that include collagen and an anti-thrombotic glycan is provided. In addition, a method of making the synthetic scaffold is provided. The synthetic scaffold is sufficiently stable to facilitate ingrowth and proliferation of biological cells. The synthetic scaffold also degrades at a rate that makes it suitable for use as a temporary cell support for use in wound healing applications.

### CROSS REFERENCE TO RELATED APPLICATIONS

This application claims priority to U.S. Provisional Patent Application No. 61/922,617, filed Dec. 31, 2013, the disclosure of which is incorporated by reference in its entirety herein.

### BACKGROUND

The skin is the largest organ in the body, covering the entire exterior of the body and forming about 8% of the total body mass. The skin serves as a self-renewing and self-repairing protective barrier between the body and the external environment. The protective effects include resistance to mechanical, chemical, osmotic and thermal injury, as well as resistance to infection by microorganisms. Skin comprises extracellular matrix components that include nano or micro-diameter fibers that include interwoven collagen fibrils. The primary cell type found in the dermis layer of skin is fibroblasts. Accordingly, research efforts to produce (e.g., using electrospinning processes known in the art) suitable artificial skin substitutes are focused on biodegradable scaffolds into which fibroblasts can infiltrate and populate. The artificial bioengineered skin should adhere and integrate with a wound.

Existing bioresorbable scaffold technologies are known that facilitate the healing of chronic and acute wounds. A number of these technologies exploit the biological properties of relatively pure natural polymers such as collagen, silk, alginate, chitosan and hyaluronate extracted from animal or plant tissue.

Biodegradable and biocompatible synthetic or natural polymers have been used to produce a variety of materials for use as skin substitutes. Synthetic polymers (e.g., poly-L-lactic acid and derivatives thereof) have been used to promote skin tissue regeneration. It has been shown that the surface microstructure and chemistry of engineered skin substitutes can affect the ability of cells and tissues to attach, grow and function. Studies have shown that electrospun collagen nanofibrous matrices are able to promote wound healing.

An off-the-shelf regenerative medical device that enabled dermatologists to provide a plastic surgeon-quality repair, without the need for grafts or flaps, would be of significant advantage. Such a device would comprise a scaffold material that assists healing, by allowing the patient's own cells to migrate and proliferate within the damaged area, forming new tissue faster and with fewer complications compared to standard non-surgical interventions.

Dermal wound healing is a complex process that requires coordination of a number of biological processes including, for example, ingrowth of cells, regulation of inflammation, and rapid wound coverage to prevent infection. There remains a need for an engineered material that facilitates rapid repair of dermal tissue at a wound site.

### SUMMARY

The present disclosure generally relates to synthetic nanofibrous scaffold compositions and a method of making said nanofibrous scaffolds. In particular, the present disclosure relates to synthetic nanofibrous scaffolds comprising collagen and an antithrombolytic glycan.

In one aspect, the present disclosure provides a synthetic scaffold. The synthetic scaffold can comprise a population of nanofibers comprising collagen and an anti-thrombotic glycan. In any embodiment, the synthetic scaffold can comprise a composite nanofiber that comprises the collagen and the anti-thrombolytic glycan. In any embodiment, the anti-thrombolytic glycan can be selected from the group consisting of heparin, dermatan sulfate, sulfated galactan, sulfated fucan, dextran, dextran sulfate, a derivative of any of the foregoing anti-thrombotic glycans, and a mixture of any two or more of the foregoing anti-thrombotic glycans.

In another aspect, the present disclosure provides an article. The article can comprise the synthetic scaffold of any of the above embodiments.

In another aspect, the present disclosure provides a method of making a composite synthetic nanofibrous scaffold. The method can comprise forming an electrospinning mixture comprising collagen and antithrombotic glycan, and electrospinning the electrospinning mixture to form a synthetic nanofibrous scaffold. In any embodiment of the method, forming the electrospinning mixture can comprise adding an active agent to the electrospinning mixture.

A “synthetic nanofibrous scaffold”, as used herein, refers to an artificial structure capable of supporting three-dimensional tissue formation. The structure comprises fibers having a fiber diameter of less than 1 micron, is highly porous, and does not substantially inhibit growth of fibroblasts or substantially inhibit infiltration of fibroblasts into the structure

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, “a” fiber can be interpreted to mean “one or more” fibers.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF THE DRAWINGS

FIG. 1 is a schematic view of an electrospinning system.

FIG. 2 is a perspective view of one embodiment of an apparatus for side-by-side electrospinning.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected” and “coupled” and variations thereof are used broadly and encompass both direct and indirect connections and couplings. Further, “connected” and “coupled” are not restricted to physical or mechanical connections or couplings. It is to be understood that other embodiments may be utilized and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

Wound healing is a special biological process that is regulated in response to physiological parameters. The wound healing process can be divided into five consecutive stages: hemostasis, inflammation, cell infiltration, cell proliferation, and maturation. Synthetic extracellular matrix structures (“scaffolds”) can assist in the successful treatment of large and deep wounds, as they effectively close the wound and help to guide cells during granulation tissue formation, fibroblast-driven remodeling, angiogenesis and re-epithelization. There is a distinct need for a new generation of bioengineering solutions that can be used to restore the damaged tissue.

The present disclosure is generally related to synthetic nanofibrous scaffold compositions and a method of making said nanofibrous scaffolds. In particular, the present disclosure relates to synthetic nanofibrous scaffolds comprising collagen and an antithrombolytic glycan. The inventive scaffolds have been found to be structurally stable and suitable as a matrix onto which biological cells (e g, mammalian cells) can attach and, yet, degrade at a rate that is suitable for use as a temporary in vivo cell support to facilitate wound healing.

Electrospinning can be performed by any means known in the art (see, for example, U.S. Pat. No. 6,110,590; which is incorporated herein by reference in its entirety). For example, a steel capillary tube with a 1.0 to 2.0 mm internal diameter tip is mounted on an adjustable, electrically insulated stand. The capillary tube is generally maintained at a high electric potential and mounted in the parallel plate geometry. The capillary tube may be connected to one or more syringes filled with a liquid containing the polymer components of the synthetic scaffold. A constant volume flow rate is usually maintained using a syringe pump, set to keep the solution at the tip of the tube without dripping. As displayed in Table 1, the electric potential (10-12 kV), solution flow rate (0.014-0.032 nL/min), and the working distance between the capillary tip and the collection screen (e.g., a 5 cm×5 cm square collection screen) are adjusted so that a stable jet is obtained. Dry or wet fibers are collected by varying the distance between the capillary tip and the collection screen.

It will be known to one skilled in the art of electrospinning that changes can be made to any of the following electrospinning parameters, which will result in scaffolds having differing architectures:
Electrospinning polymer solution concentration.
Electrospinning solvent
Electrospinning voltage
Electrospinning duration
Fiber collector type, shape, or construction material
Diameter, rotation speed or length of cylindrical collector
Needle traverse distance, frequency or speed
Needle diameter, length, cross-sectional shape, or construction material
Number of needles or arrangement of needles
Needle to collector separation distance

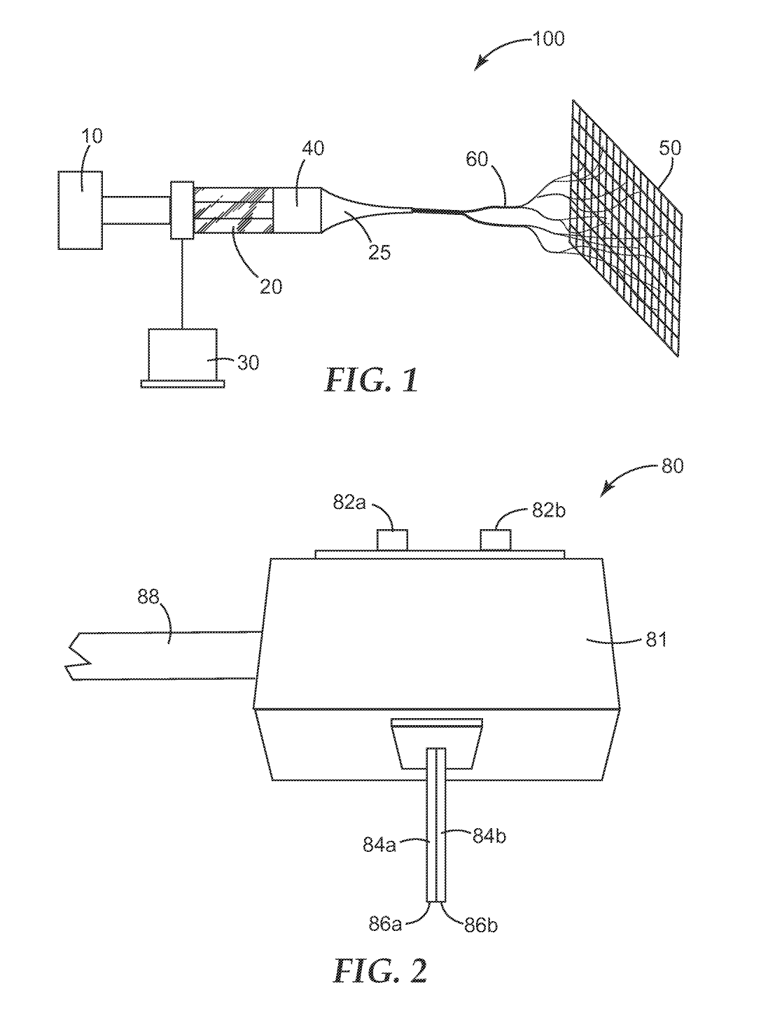

FIG. 1 shows a schematic side view of a typical electrospinning system 100. The system 100 comprises a metering pump 10 that controls the flow of the electrospinning fluid 40, a syringe 20 into which the electrospinning fluid 40 is loaded, a tip 25 operatively connected to the syringe 20 and through which the electrospinning fluid 40 is ejected during electrospinning, a high-voltage supply 30 operatively connected to the syringe 20, and a collector 50. The metering pump 10 urges the electrospinning fluid 40 out of the syringe 20 through the tip 25.

FIG. 2 shows a top perspective view of one embodiment of an apparatus 80 for performing side-by-side electrospinning. The apparatus comprises a hollow block 81 through which two fluid pathways (not shown) pass. A first fluid pathway starts at inlet 82a, passes through block 81 and tip 84a, and ends at orifice 86a. A second fluid pathway starts at inlet 82b, passes through block 81 and tip 84b, and ends at orifice 86b. The orifices 86a and 86b are juxtaposed such that a first fluid passing through the first fluid pathway contacts a second fluid simultaneously passing through the second fluid pathway as both fluids are ejected from orifices 86a and 86b, respectively.

TABLE 1
Exemplary electrospinning parameters for a collagen/Heparin
sulfate electrospinning mixture.

| | Potential-Ground | |
|---|---|---|
| Collagen/Heparin Sulfate | Working Distance | Injection Rate |
| (w/w) | (cm) | (mL/hr) |
| 98:2 | 12 | 3 |

A collection plate or a collection screen suitable for collecting collagen/antithrombolytic fibers can be a wire mesh or a polymeric mesh. Alternatively, the collection screen is an aluminum foil. The aluminum foil can be coated with Teflon fluid to make peeling off the fibers easier. One skilled in the art will be able to readily select other means of collecting the fiber solution as it travels through the electric field. As is described in more detail below, the electric potential difference between the capillary tip and the aluminum foil counter electrode may be gradually increased, however, one skilled in the art should be able to adjust the electric potential to achieve suitable jet stream.

An embodiment of the present invention provides for composite nanofibrous scaffolds with potential utility for wound dressings prepared utilizing a blend of collagen/antithrombolytic glycan (e.g., heparin sulfate) in one-fluid or two-fluid (e.g., coaxial or side-by side configurations) electrospinning techniques. Two-fluid electrospinning techniques are described in Rutledge et al., Advanced Drug Delivery Reviews, Vol. 59, pp 1384-1391 (2007); which is incorporated herein by reference in its entirety.

The present disclosure thus provides for processes for production of synthetic nanofibrous scaffold mats. The process comprises the steps of forming an electrospin mixture comprising collagen and antithrombotic glycan, and electrospinning the electrospin mixture to form a synthetic nanofibrous scaffold. The resulting synthetic nanofibrous scaffold is a composite scaffold. In any embodiment, forming the electrospin mixture can comprise mixing the collagen and the antithrombolytic glycan (e.g., heparin sulfate) in a suitable solvent (e.g., trifluoroethane (TFA)).

In any embodiment, the mass ratio of collagen:antithrombolytic glycan in the electrospinning mixture can be adjusted to provide certain desirable features (e.g., tensile strength, biocompatibility, biodegradability, oxygen vapor transfer rate, hydration rate, and improved cell proliferation on the scaffold. In any embodiment, the mass ratio of collagen:antithrombolytic glycan in the electrospinning mixture is about 90:10. In any embodiment, the mass ratio of collagen:antithrombolytic glycan in the electrospinning mixture is about 92:8. In any embodiment, the mass ratio of collagen:antithrombolytic glycan in the electrospinning mixture is about 94:6. In any embodiment, the mass ratio of collagen:antithrombolytic glycan in the electrospinning mixture is about 95:5. In any embodiment, the mass ratio of collagen:antithrombolytic glycan in the electrospinning mixture is about 96:4. In any embodiment, the mass ratio of collagen:antithrombolytic glycan in the electrospinning mixture is about 98:2. In any embodiment, the mass ratio of collagen:antithrombolytic glycan in the electrospinning mixture is about 99:1.

In any embodiment of the method, forming an electrospin mixture comprising collagen and antithrombotic glycan comprises forming a first component, forming a second component, and mixing the first component and the second component to form the electrospin mixture. In any embodiment, the first component may comprise a first solvent and the second component may comprise an aqueous second solvent that is substantially miscible with the first solvent. A nonlimiting example of this embodiment comprises forming a first component comprising collagen mixed in a first solvent (e.g., TFA), forming a second component comprising heparin sulfate mixed in a second solvent (water), and mixing the first component and the second component to form the electrospin mixture.

The first solvent should be miscible with water or aqueous solution substantially comprising water. In any embodiment, the first solvent can be an organic solvent (e.g., trifluoroethane) or an acid (e.g., acetic acid, formic acid).

In any embodiment of the method, the first component can be mixed with the second component to form the electrospin mixture prior to electrospinning the mixture. For example, the first and second components can be formed in separate containers, then the contents of the separate containers can be brought into contact with each other and mixed in a common container before loading the mixture into a part of the electrospin apparatus (e.g., before loading the mixture into a syringe that drives the electrospin process). Alternatively, the first and second components can be brought into contact and mixed in a part (e.g., a syringe) of the electrospin apparatus before the syringe is used in the electrospinning process.

In any embodiment, the first component can be mixed with the second component during the electrospinning process. For example, the electrospinning apparatus can be configured with an apparatus configured for side-by-side delivery of electrospinning fluids (e.g., the apparatus 80 illustrated in FIG. 2, available from NaBond Technologies Co., Ltd.; Hong Kong). In this configuration, the first component can enter the apparatus via a first flow path and the second component can enter the apparatus via a second flow path and the two components are intermixed as they are expelled from the spinneret as a composite mixture comprising collagen and glycan.

In any embodiment of the method, forming the electrospinning mixture comprises adding an active agent to the electrospinning mixture. The active agent can be any active agent disclosed herein. In this embodiment, the active agent may be disposed on the surface of the fibers of the synthetic nanofibrous scaffolds, it may be embedded in the fibers of the synthetic nanofibrous scaffolds, or it may be disposed on the surface and embedded in the fibers of the synthetic nanofibrous scaffolds. Accordingly, the active agent can be delivered into a wound site with the synthetic nanofibrous scaffolds where a portion, or all, of the active agent may be released upon contact of the synthetic nanofibrous scaffolds with the tissue and/or interstitial fluid of the wound site. Alternatively or additionally, a portion, or all, of the active agent may be released over a period of time (e.g., several minutes, several hours, several days, or several weeks) into the tissue and/or interstitial fluid of the wound site.

In any embodiment, the active agent can be added into the electrospinning mixture via a first component of the present disclosure (i.e., the active agent may be mixed with the collagen in the first component). Alternatively or additionally, in any embodiment, the active agent can be added into the electrospinning mixture via a second component of the present disclosure (i.e., the active agent may be mixed with the antithrombolytic glycan in the second component).

In any embodiment of the method, an active agent can be coated onto the synthetic scaffold. The active agent can be any active agent disclosed herein. The active agent can be coated using coating processes known in the art (e.g., dip-coating, kiss-coating, spray coating).

Additional biocompatible material may also be blended into the scaffolds, such as polyethylene glycol, fibronectin, keratin, polyaspartic acid, polylysine, alginate, chitosan, chitin, hyaluronic acid, pectin, polycaprolactone, polylactic acid, polyglycolic acid, polyhydroxyalkanoates, polyanhydrides, glycerol, and other biocompatible polymers. Additionally, or alternatively, the composite nanofibrous scaffolds may be mixed with hydroxyapatite particles.

The scaffold-embedded active agents or biological materials may be suitable for long term storage and stabilization of the cells and/or active agents. Cells and/or active agents, when incorporated in the composite nanofibrous scaffolds, may be stable for extended periods at room temperature (i.e., 22° C. to 25° C.) and at body temperature (37° C.).

The composite nanofibrous scaffolds embedded active agents (e.g., therapeutic agents) or biological materials are suitable for a biodelivery device. The use of collagen as a biodelivery material is disclosed, for example, in Ruszczak et al., Adv Drug Del Rev, vol. 55, pp 1679-1698 (2003), which is incorporated herein by reference in its entirety. Some embodiments of the present invention relate to the utility of scaffold-embedded therapeutic agents or biological materials as drug delivery systems for potential utility in medical implants, tissue repairs and for medical device coatings.

The synthetic nanofibrous scaffold structure enables the biodelivery vehicle to have a controlled release. Controlled release permits dosages to be administered over time, with controlled release kinetics. In some instances, delivery of the therapeutic agent or biological material is continuous to the site where treatment is needed, for example, over several weeks. Controlled release over time, for example, over several days or weeks, or longer, permits continuous delivery of the therapeutic agent or biological material to obtain preferred treatments. The controlled delivery vehicle is advantageous because it protects the therapeutic agent or biological material from degradation in vivo in body fluids and tissue, for example, by proteases.

Controlled release of the bioactive agent from the synthetic nanofibrous scaffold may be designed to occur over time, for example, for greater than about 12 hours or 24 hours, inclusive; greater than 1 month or 2 months or 5 months, inclusive. The time of release may be selected, for example, to occur over a time period of about 12 hours to 24 hours, or about 12 hours to 1 week. In another embodiment, release may occur for example on the order of about 1 month to 2 months, inclusive. The controlled release time may be selected based on the condition treated. For example, a particular release profile may be more effective where consistent release and high local dosage are desired.

In any embodiment, a therapeutic agent can be coated on to the composite nanofibrous scaffolds with a pharmaceutically acceptable carrier. Any pharmaceutical carrier can be used that does not dissolve the matrix. The therapeutic agents may be present as a liquid, a finely divided solid, or any other appropriate physical form. In these embodiments, optionally, the matrix will include one or more additives, such as diluents, carriers, excipients, stabilizers or the like.

The amount of therapeutic agent will depend on the particular drug being employed and medical condition being treated. For example, the amount of drug may represent about 0.001% to about 70%, or about 0. 001% to about 50%, or about 0.001% to about 20% by weight of the material. Upon contact with body fluids the drug will be released.

The synthetic nanofibrous scaffolds can be further modified after fabrication. For example, the scaffolds can be coated with bioactive substances that function as receptors or chemoattractors for a desired population of cells. The coating can be applied through absorption or chemical bonding.

In one embodiment, the synthetic nanofibrous scaffolds of the present invention may contain at least one therapeutic agent. To form these materials, the electrospin mixture is mixed with a therapeutic agent prior to forming the composite nanofibrous scaffolds, or is loaded into the material after it is formed. The variety of different therapeutic agents that can be used in conjunction with the biomaterials of the present invention is vast.

In general, therapeutic agents which may be administered via the pharmaceutical compositions of the invention include, without limitation: antiinfectives such as antibiotics and antiviral agents; chemotherapeutic agents (e.g., anticancer agents); anti-rejection agents; analgesics and analgesic combinations; anti-inflammatory agents; hormones such as steroids; cell attachment mediators, such as the peptide containing variations of the “RGD” integrin binding sequence known to affect cellular attachment, biologically active ligands, and substances that enhance or exclude particular varieties of cellular or tissue ingrowth such as bone morphogenic proteins (e.g., BMPs 1-7), bone morphogenic-like proteins (e.g., GFD-5, GFD-7, and GFD-8), epidermal growth factor (EGF), fibroblast growth factor (e.g., FGF 1-9), platelet derived growth factor (PDGF), insulin like growth factor (IGF-I and IGF-II), transforming growth factors (e.g., TGF-b I-III), TGF-, YIGSR peptides, glycosaminoglycans (GAGs), hyaluronic acid (HA), integrins, selectins and cadherins; vascular endothelial growth factor (VEGF); and other naturally derived or genetically engineered proteins, polysaccharides, glycoproteins, or lipoproteins. Growth factors are known in the art.

The active agent can represent any material capable of being embedded in the collagen-containing scaffold. For example, the agent may be a therapeutic agent, or a biological material, such as cells (including stem cells), proteins, peptides, nucleic acids (e.g., DNA, RNA, siRNA), nucleic acid analogs, nucleotides, oligonucleotides, peptide nucleic acids (PNA), aptamers, antibodies or fragments or portions thereof (e.g., paratopes or complementarity-determining regions), antigens or epitopes, hormones, hormone antagonists, growth factors or recombinant growth factors and fragments and variants thereof, cell attachment mediators (such as RGD), cytokines, enzymes, small molecules, drugs, dyes, amino acids, vitamins, antioxidants, antibiotics or antimicrobial compounds, anti-inflammation agents, antifungals, viruses, antivirals, toxins, prodrugs, chemotherapeutic agents, or combinations thereof. The agent may also be a combination of any of the abovementioned agents. Encapsulating either a therapeutic agent or biological material, or the combination of them, is desirous because the encapsulated product can be used for numerous biomedical purposes.

Exemplary enzymes suitable for use herein include, but are not limited to, peroxidase, lipase, amylose, organophosphate dehydrogenase, ligases, restriction endonucleases, ribonucleases, DNA polymerases, glucose oxidase, laccase, and the like. Interactions between components may also be used to functionalize silk fibroin through, for example, specific interaction between avidin and biotin. The active agents can also be the combinations of any of the enzymes listed above.

In some embodiments, the active agent may also be an organism such as a fungus, plant, animal, bacterium, or a virus (including bacteriophage). Moreover, the active agent may include neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins and radiopharmaceuticals.

Exemplary cells suitable for use herein may include, but are not limited to, progenitor cells or stem cells, smooth muscle cells, skeletal muscle cells, cardiac muscle cells, epithelial cells, endothelial cells, urothelial cells, fibroblasts, myoblasts, oscular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, and precursor cells. The active agents can also be the combinations of any of the cells listed above.

Exemplary antibodies that may be incorporated in the synthetic nanofibrous scaffolds of the present disclosure include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab. The active agents can also be the combinations of any of the antibodies listed above.

Exemplary antibiotic agents include, but are not limited to, actinomycin; aminoglycosides (e.g., neomycin, gentamicin, tobramycin); β-lactamase inhibitors (e.g., clavulanic acid, sulbactam); glycopeptides (e.g., vancomycin, teicoplanin, polymixin); ansamycins; bacitracin; carbacephem; carbapenems; cephalosporins (e.g., cefazolin, cefaclor, cefditoren, ceftobiprole, cefuroxime, cefotaxime, cefipeme, cefadroxil, cefoxitin, cefprozil, cefdinir); gramicidin; isoniazid; linezolid; macrolides (e.g., erythromycin, clarithromycin, azithromycin); mupirocin; penicillins (e.g., amoxicillin, ampicillin, cloxacillin, dicloxacillin, flucloxacillin, oxacillin, piperacillin); oxolinic acid; polypeptides (e.g., bacitracin, polymyxin B); quinolones (e.g., ciprofloxacin, nalidixic acid, enoxacin, gatifloxacin, levaquin, ofloxacin, etc.); sulfonamides (e.g., sulfasalazine, trimethoprim, trimethoprim-sulfamethoxazole (cotrimoxazole), sulfadiazine); tetracyclines (e.g., doxycyline, minocycline, tetracycline, etc.); monobactams such as aztreonam; chloramphenicol; lincomycin; clindamycin; ethambutol; mupirocin; metronidazole; pefloxacin; pyrazinamide; thiamphenicol; rifampicin; thiamphenicl; dapsone; clofazimine; quinupristin; metronidazole; linezolid; isoniazid; piracil; novobiocin; trimethoprim; fosfomycin; fusidic acid; or other topical antibiotics. Optionally, the antibiotic agents may also be antimicrobial peptides such as defensins, magainin and nisin; or lytic bacteriophage. The antibiotic agents can also be the combinations of any of the agents listed above.

The electrospun collagen/glycan mats prepared by the processes of the present disclosure exhibit good structural, morphological, biofunctional and biocompatible properties suitable for biomaterial application, such as wound dressing. For example, the resulting collagen/glycan mats (e.g., a collagen/heparin sulfate nanofibrous scaffolds) of the present disclosure degrade more than about 27% (wt) in about 10 days. In addition, the resulting collagen/glycan mats (e.g., a collagen/heparin sulfate nanofibrous scaffolds) of the present disclosure degrade more than about 70% (wt) in about 20 days. In addition, the resulting collagen/glycan mats (e.g., a collagen/heparin sulfate nanofibrous scaffolds) of the present disclosure degrade more than about 92% (wt) in about 30 days. Normal human skin regenerates in about 21 days. Thus, the degradation rate of the synthetic nanofibrous scaffolds of the present disclosure coincides with the amount of time required for normal regeneration of skin tissue.

The synthetic nanofibrous scaffolds produced by the processes of the present invention may be used in a variety of medical applications such as wound closure systems, including vascular wound repair devices, hemostatic dressings, full thickness burn wound dressing, patches and glues, sutures, drug delivery and in tissue engineering applications, such as, for example, scaffolding, ligament prosthetic devices and in products for long-term or bio-degradable implantation into the human body. An exemplary tissue engineered scaffold is a non-woven network of electrospun fibers.

Additionally, these biomaterials can be used for organ repair replacement or regeneration strategies that may benefit from these unique scaffolds, including but are not limited to, spine disc, cranial tissue, dura, nerve tissue, liver, pancreas, kidney, bladder, spleen, cardiac muscle, skeletal muscle, tendons, ligaments, and breast tissues.

In another aspect, the present disclosure provides synthetic nanofibrous scaffolds made according to the methods disclosed herein. The scaffolds comprise a population of nanofibers comprising collagen and an anti-thrombotic glycan (e.g., an antithrombolytic glycan selected from the group consisting of heparin, dermatan sulfate, sulfated galactan, sulfated fucan, dextran, dextran sulfate, a derivative of any of the foregoing anti-thrombotic glycans, and a mixture of any two or more of the foregoing anti-thrombotic glycans).

In any embodiment, the mass ratio of collagen:antithrombolytic glycan in the synthetic nanofibrous scaffold is about 90:10. In any embodiment, the mass ratio of collagen:antithrombolytic glycan in the synthetic nanofibrous scaffold is about 92:8. In any embodiment, the mass ratio of collagen:antithrombolytic glycan in the synthetic nanofibrous scaffold is about 94:6. In any embodiment, the mass ratio of collagen:antithrombolytic glycan in the synthetic nanofibrous scaffold is about 95:5. In any embodiment, the mass ratio of collagen:antithrombolytic glycan in the synthetic nanofibrous scaffold is about 96:4. In any embodiment, the mass ratio of collagen:antithrombolytic glycan in the synthetic nanofibrous scaffold is about 98:2. In any embodiment, the mass ratio of collagen:antithrombolytic glycan in the synthetic nanofibrous scaffold is about 99:1.

In any embodiment, the synthetic nanofibrous scaffold produced by a method of the present disclosure comprises aligned fibers (i.e., the scaffold is an aligned scaffold). Alternatively, in any embodiment, the synthetic nanofibrous scaffold produced by a method of the present disclosure comprises random, non-aligned fibers (i.e., the scaffold is a random fibrous scaffold).

In any embodiment, a synthetic nanofibrous scaffold produced by a method of the present disclosure comprises an active agent, as described herein. The active agent may be disposed on the surface of the nanofibers and/or may be embedded in the nanofibers.

In any embodiment, a synthetic nanofibrous scaffold of the present disclosure facilitates ingrowth of biological cells. In any embodiment, a synthetic nanofibrous scaffold of the present disclosure facilitates proliferation of biological cells. The biological cells can be selected from the group consisting of stem cells, pluripotent stem cells, committed stem cells, embryonic stem cells, adult stem cells, bone marrow stem cells, adipose stem cells, umbilical cord stem cells, dura mater stem cells, precursor cells, differentiated cells, osteoblasts, myoblasts, neuroblasts, fibroblasts, ghoblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, normal cells, cancer cells, Schwann cells, and neurons.

In any embodiment, a synthetic nanofibrous scaffold of the present disclosure further comprises a metabolically-active biological cell disposed therein or thereon. In any embodiment, a synthetic nanofibrous scaffold of the present disclosure further comprises a metabolically-active biological cell attached thereto. The metabolically active cell may be attached via biopolymers (e.g., extracellular matrix polymers.

In another aspect, the present disclosure provides an article comprising any embodiment of the synthetic nanofibrous scaffold described herein. The article can comprise, for example, a medical device (e.g., an implantable medical device). In any embodiment, the article can comprise a wound dressing. In any embodiment of the article, the synthetic nanofibrous scaffold is attached thereto (e.g., detachably attached via a biodegradable polymer).

### Exemplary Embodiments

Embodiment A is a synthetic scaffold, the synthetic scaffold comprising a population of nanofibers comprising collagen and an anti-thrombotic glycan.

Embodiment B is the synthetic scaffold of Embodiment A, wherein the synthetic scaffold comprises a composite nanofiber that comprises the collagen and the anti-thrombolytic glycan.

Embodiment C is the synthetic scaffold of Embodiment A or Embodiment B, wherein the antithrombotic glycan is selected from the group consisting of heparin, dermatan sulfate, sulfated galactan, sulfated fucan, dextran, dextran sulfate, a derivative of any of the foregoing anti-thrombotic glycans, and a mixture of any two or more of the foregoing anti-thrombotic glycans.

Embodiment D is the synthetic scaffold of any one of the preceding Embodiments, wherein the synthetic scaffold has a mass ratio of collagen:glycan of about 90:10 to about 99 to 1.

Embodiment E is the synthetic scaffold of any one of the preceding Embodiments, wherein the synthetic scaffold is an aligned fibrous scaffold.

Embodiment F is the synthetic scaffold of any one of Embodiments A through E, wherein the synthetic scaffold is a random fibrous scaffold.

Embodiment G is the synthetic scaffold of any one of the preceding Embodiments, wherein the synthetic scaffold facilitates the proliferation of biological cells selected from the group consisting of stem cells, pluripotent stem cells, committed stem cells, embryonic stem cells, adult stem cells, bone marrow stem cells, adipose stem cells, umbilical cord stem cells, dura mater stem cells, precursor cells, differentiated cells, osteoblasts, myoblasts, neuroblasts, fibroblasts, ghoblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, normal cells, cancer cells, Schwann cells, and neurons.

Embodiment H is the synthetic scaffold of any one of the preceding Embodiments, further comprising a metabolically-active biological cell attached thereto.

Embodiment I is the synthetic scaffold of any one of the preceding Embodiments, further comprising an active agent.

Embodiment J is the synthetic scaffold of Embodiment I, wherein the active agent is a therapeutic agent or a biological material, selected from the group consisting of cells, proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, prodrugs, chemotherapeutic agents, small molecules, drugs, and combinations thereof.

Embodiment K is the synthetic scaffold of Embodiment J, wherein the active agent is a cell selected from the group consisting of progenitor cells or stem cells, smooth muscle cells, skeletal muscle cells, cardiac muscle cells, epithelial cells, endothelial cells, urothelial cells, fibroblasts, myoblasts, oscular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, precursor cells, and combinations thereof.

Embodiment L is the synthetic scaffold of Embodiment K, wherein the active agent further comprises a cell growth media.

Embodiment M is the synthetic scaffold of Embodiment K, wherein the active agent is an antibiotic.

Embodiment N is an article comprising the synthetic scaffold of any one of the preceding Embodiments.

Embodiment O is a method of making a composite synthetic nanofibrous scaffold, the method comprising:

forming an electrospinning mixture comprising collagen and antithrombotic glycan; and

electrospinning the electrospinning mixture to form a synthetic nanofibrous scaffold.

Embodiment P is the method of Embodiment O, wherein forming an electrospinning mixture comprising collagen and antithrombotic glycan comprises:

forming a first component, wherein forming a first component comprises mixing collagen with a first solvent;

forming a second component, wherein forming a second component comprises mixing the antithrombotic glycan in a second solvent; and

mixing the first component with the second component to form the electrospinning mixture.

Embodiment Q is the method of Embodiment O or embodiment P, wherein the electrospinning mixture comprises the collagen and the antithrombotic glycan in a mass ratio of about 90:10, respectively, to about 99 to 1, respectively.

Embodiment R is the method of any one of Embodiments O through Q, wherein forming the electrospinning mixture occurs prior to electrospinning the electrospinning mixture.

Embodiment S is the method of any one of Embodiments O through Q, wherein forming the electrospinning mixture occurs while electrospinning the electrospinning mixture.

Embodiment T is the method of any one of Embodiments O through S, wherein forming the electrospinning mixture comprises adding an active agent to the electrospinning mixture.

Embodiment U is the method of Embodiment T as dependent on Embodiment P, wherein adding an active agent to the electrospinning mixture comprises adding the active agent to the first component.

Embodiment V is the method of Embodiment T as dependent on Embodiment P, wherein adding an active agent to the electrospinning mixture comprises adding the active agent to the second component.

Embodiment W is the method of any one of Embodiments O through V, further comprising coating an active agent onto the synthetic scaffold.

Embodiment X is the method of Embodiment T, wherein the active agent is a therapeutic agent or a biological material, selected from the group consisting of cells, proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, prodrugs, chemotherapeutic agents, small molecules, drugs, and combinations thereof.

Embodiment Y is the method of Embodiment X, wherein the active agent further comprises a cell culture medium.

Advantages and embodiments of this disclosure are further illustrated by the following examples, but the particular materials and amounts thereof recited in these examples, as well as other conditions and details, should not be construed to unduly limit this disclosure. All materials are commercially available or known to those skilled in the art unless otherwise stated or apparent.

### EXAMPLES

Materials

TABLE 1
Materials used in the Examples.

| Name | Material | Source |
|---|---|---|
| Cell culture | Medium 106 with supplement | Life Technologies; Grand |
| medium | (Catalog Number M106500) | Island, NY |
| Collagen | Collagen; Catalog Number | Elastin Products |
| | C806 | Company, |
| | | Owensville, Missouri |
| Dextran | Dextran; Catalog Number | Sigma Chemical Co., St |
| | 09184 | Louis, MO |
| Gelatin | Gelatin; Catalog Number | Sigma Chemical Co. |
| | G9382 | |
| Heparin Sulfate | Heparin Sulfate; Catalog | Sigma Chemical Co. |
| | Number H7640 | |
| HFIP | Hexafluoro-2-propanol | |
| HDFa | Human dermal fibroblasts, | Life Technologies |
| | adult; Catalog Number | |
| | C0135C | |
| HEKa | Human Epidermal | Life Technologies |
| | keratinocytes, adult; Catalog | |
| | Number C0215C | |
| MTS Assay Kit | Catalog Number G5421 | Promega Corp.; |
| | | Madison, WI |
| phosphate | Catalog Number D8662 | Sigma Chemical Co. |
| buffered saline | | |
| (PBS) | | |
| TFE | Trifluoroethane; Catalog | Sigma Chemical Co. |
| | Number T63002 | |

Electrospinning Conditions

### Reference Examples 1-4

Reference Examples 1-4 were prepared by electrospinning the materials listed in Table 2 under the conditions reported in Table 2. The equipment was set up as shown in FIG. 1. The high-voltage power supply (part number CZE 1000R) obtained from Spellman High Voltage Electronics Corporation, Hauppauge, N.Y. The syringe pump (part number AS40A was obtained from Baxter (Deerfield, Ill.). The tip was a blunt stainless steel needle (½″×18 gauge) obtained from Small Parts, Inc. (Logansport, Ind.). Polymer solutions were prepared as specified in Table 2. The solutions were transferred to the syringe and loaded on the syringe pump. Each solution was electrospun according to the conditions specified in Table 2. The resulting nanofibrous scaffold was collected on a 5 cm×5 cm square non-rotating collector. The typical thickness of the resulting scaffolds was 0.5 mm.

### Example 1

Example 1 was prepared by electrospinning the materials listed in Table 2 under the conditions reported in Table 2. The equipment was set up as shown in FIG. 1 with the exception that the apparatus 80 (available from NaBond Technologies Co., Ltd.; Hong Kong) of FIG. 2 was connected to two separate syringes, one containing collagen dissolved in TFE and the other containing heparin sulfate dissolved in water. Polymer solutions were prepared as specified in Table 2. The solutions (each of them containing 12.5% (wt/vol) of the respective polymer) were transferred to individual syringes and loaded on the syringe pump, which controlled the ration of the final mixture delivered to the tip. The electrospinning conditions are shown in Table 2. The resulting nanofibrous scaffold was collected on a 5 cm×5 cm square non-rotating collector. The typical thickness of the resulting scaffolds was 0.5 mm.

TABLE 2
Polymer solutions and electrospinning conditions for
Reference Examples 1-4 and Example 1.

| | | Electrospinning Conditions | | | | |
|---|---|---|---|---|---|---|
| | | | Flow | Work | | |
| | | Voltage | Rate | Distance | | Concentration % |
| | Materials | (kV) | (mL/hr) | (cm) | Solvent | (Wt/Vol) |
| Reference | Dextran | 20 | 2 | 15 | Water:EtOH | 8 |
| Example 1 | | | | | (80%:20%) | |
| Reference | Collagen | 25 | 3 | 12 | TFE | 14 |
| Example 2 | | | | | | |
| Reference | Gelatin | 20 | 2 | 10 | HFIP | 10 |
| Example 3 | | | | | | |
| Reference | Dextran/Heparin | 20 | 0.5 | 15 | Water:EtOH | 10 |
| Example 4 | Sulfate | | | | (80%:20%) | |
| | (98%:2%) | | | | | |
| Example 1 | Collagen/Heparin | 25 | 3 | 12 | TFE:Water | 12 |
| | Sulfate | | | | | |
| | (98%:2% | | | | | |

The resulting synthetic nanofibrous scaffolds were characterized by scanning electron microscopy. The electron micrographs showed randomly-oriented fiber meshes comprising a plurality of submicron-diameter fibers.

Degradation Testing

The material biodegradation profile is measured from the electrospun material mass loss in PBS solution.

The electrospun materials were frozen in −80° C. overnight and then lyophilized for at least 12 hours. The dry material was weighed. Each sample had a weight of 0.5-1 gram. The samples were individually immersed in 1 mL of PBS in individual sterile tubes and placed in an incubator set at 37° C. The PBS was changed every 3 days. After the specified periods of incubation time, three samples were taken out of the PBS solution. The residues are centrifuged in an Allegra 6R centrifuge (Beckman Coulter Inc.; Brea, Calif.) for 10 mins at 1000 rpm (183×g). After centrifugation, the supernatant was removed and the samples were frozen −80° C. overnight, lyophilized for at least 12 hours and weighed again. The results are shown in Table 3. The weight loss was calculated by the flowing equation:

Weight loss=(W₀−Wₜ)/Wₜ×100%,

where W₀=initial weight of sample and Wₜ=weight of sample at specified time points.

TABLE 3
Weight loss of synthetic nanofibrous scaffolds after
immersion in PBS at 37° C. for predetermined periods
of time. The results are the average weight loss for three
identical samples at each specified time point.

| | Degradation of Synthetic Nanofibrous Scaffolds | | |
|---|---|---|---|
| SAMPLE | 10 days | 20 days | 30 days |
| Reference | 36.1% | 76.3% | 95.2% |
| Example 1 | | | |
| Reference | 22.8% | 69.8% | 96.1% |
| Example 2 | | | |
| Reference | 30.2% | 82.1% | 92.5% |
| Example 3 | | | |
| Reference | 38.9% | 80.4% | 94.4% |
| Example 4 | | | |
| Example 1 | 27.2% | 70.2% | 92.7% |

Cytotoxicity Testing

Electrospun materials were sterilized in 70% ethanol for 30 minutes at room temperature followed by 3 rinses (15 mL per rinse) in phosphate buffered saline (PBS). The materials were incubated at 37° C. for 30 min in 5 mL culture medium prior to testing. Samples (6 mm×6 mm×0.5 mm) of synthetic scaffolds were placed at the center of the wells of 24-well plates that containing confluent monolayer of human dermal fibroblasts, adults (HDFa) (Life Technologies). Wells containing cells only (i.e., no synthetic scaffold) served as the control.

Cells were cultured at 37° C. in a 5% CO₂ incubator. Cell culture medium (Medium 106 with supplement from Life Technologies) was changed every 3 days. The MTS assay (Promega) was performed at day 1, 3 and 7 to determine cell viability. The results (shown in Table 3) were reported as the percentage of viable cells relative to the controls (n=5). Samples that showed cell viability that was greater than 70% of the control indicated good biocompatibility of the synthetic nanofibrous scaffolds. Table 4 shows that the synthetic nanofibrous scaffolds of Example 1 had at least 92% viability, relative to the controls, at every time point tested, indicating very good biocompatibility.

TABLE 4
Biocompatibility test results. All data points represent the average of 5
samples at each specified time point.

| | CELL VIABILITY (% OF CONTROL) | | |
|---|---|---|---|
| MATERIALS | 1 DAY | 3 DAYS | 30 DAYS |
| Reference | 88.3% | 90.5% | 92% |
| Example 1 | | | |
| Reference | 91.7% | 93.3% | 95.1% |
| Example 2 | | | |
| Reference | 85.2% | 90.1% | 92.2% |
| Example 3 | | | |
| Reference | 89.9% | 94.2% | 90.8% |
| Example 4 | | | |
| Example 1 | 94.1% | 92.2% | 95.9% |

Evaluation of Cell Growth on Nanofibrous Structures

HEKa and HDFa were cultured for 2 days in cell culture medium containing synthetic nanofibrous scaffold samples in order to study the cell attachment and proliferation on the nanofibrous scaffolds. The size of the scaffold material used in these tests was (1 cm×1 cm×0.5 mm) Cells were cultured in tissue culture polystyrene flasks at 37° C. in a 5% CO₂ atmosphere. Before cell seeding, the scaffold material was soaked in the culture medium in the incubator 37° C. for 6 hrs. Tissue culture flasks (75 cm²) containing a confluent monolayer of cells were washed three times with PBS and then trypsinized with 3 ml Typsin solution (TrpLE Express, Invitrogen Life Technologies) at room temperature for 5 minutes until cells became rounded and suspended. The trypsinzed cell suspension was transferred to a centrifuge tube and centrifuged 5 minutes at 1000 rpm, as described above. After centrifugation, the supernatant was removed and the cells were pipetted onto the surface of the nanofibrous scaffold samples at a density of 5×10⁵ cells/cm².

The samples were observed by scanning electron microscopy using a method similar to that described in Venugopal et al. (In Vitro Culture of Human Dermal Fibroblasts on Electrospun Polycaprolactone Collagen Nanofibrous Membrane; Artificial Organs 2006; 30(6):440-446), which is incorporated herein by reference in its entirety. After 2 days of the cell growth on the scaffold samples, the samples containing HDF cells were washed with PBS to remove nonadherent cells and then fixed in 4% glutaraldehyde for 1 h at room temperature. After fixing the samples, they were dehydrated through a series of graded alcohol, and finally dried in hexamethyldisilazan overnight to maintain the normal cell morphology. Dried samples were sputter-coated with gold and observed under the scanning electron microscope (SEM) at an accelerating voltage of 10 kV. The SEM images showed that cells grow very well on the electrospun scaffolds.

The samples were also observed microscopically after histological staining. For histological evaluation, samples were imbedded in paraffin, sectioned at 0.5 μm and stained with a hematoxylin and eosin (H&E) staining protocol. The H&E stained samples also showed the cells adhered very well on the scaffolds and proliferated while in contact with the scaffolds.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

Various modifications may be made without departing from the spirit and scope of the invention. These and other embodiments are within the scope of the following claims.

1. A synthetic scaffold, the synthetic scaffold comprising a population of nanofibers comprising collagen and an anti-thrombotic glycan.

2. The synthetic scaffold of claim 1, wherein the synthetic scaffold comprises a composite nanofiber that comprises the collagen and the anti-thrombolytic glycan.

3. The synthetic scaffold of claim 1, wherein the antithrombotic glycan is selected from the group consisting of heparin, dermatan sulfate, sulfated galactan, sulfated fucan, dextran, dextran sulfate, a derivative of any of the foregoing anti-thrombotic glycans, and a mixture of any two or more of the foregoing anti-thrombotic glycans.

4. The synthetic scaffold of claim 1, wherein the synthetic scaffold has a mass ratio of collagen:glycan of about 90:10 to about 99 to 1.

5. The synthetic scaffold of claim 1, wherein the synthetic scaffold is an aligned fibrous scaffold.

6. The synthetic scaffold of claim 1, wherein the synthetic scaffold is a random fibrous scaffold.

7. The synthetic scaffold of claim 1, wherein the synthetic scaffold facilitates the proliferation of biological cells selected from the group consisting of stem cells, pluripotent stem cells, committed stem cells, embryonic stem cells, adult stem cells, bone marrow stem cells, adipose stem cells, umbilical cord stem cells, dura mater stem cells, precursor cells, differentiated cells, osteoblasts, myoblasts, neuroblasts, fibroblasts, ghoblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, normal cells, cancer cells, Schwann cells, and neurons.

8. The synthetic scaffold of claim 1, further comprising a metabolically-active biological cell attached thereto.

9. The synthetic scaffold of claim 1, further comprising an active agent.

10. The synthetic scaffold of claim 9, wherein the active agent is a therapeutic agent or a biological material, selected from the group consisting of cells, proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, prodrugs, chemotherapeutic agents, small molecules, drugs, and combinations thereof.

11. The synthetic scaffold of claim 10, wherein the active agent is a cell selected from the group consisting of progenitor cells or stem cells, smooth muscle cells, skeletal muscle cells, cardiac muscle cells, epithelial cells, endothelial cells, urothelial cells, fibroblasts, myoblasts, oscular cells, chondrocytes, chondroblasts, osteoblasts, osteoclasts, keratinocytes, kidney tubular cells, kidney basement membrane cells, integumentary cells, bone marrow cells, hepatocytes, bile duct cells, pancreatic islet cells, thyroid, parathyroid, adrenal, hypothalamic, pituitary, ovarian, testicular, salivary gland cells, adipocytes, precursor cells, and combinations thereof.

12. An article comprising the synthetic scaffold of claim 1.

13. A method of making a composite synthetic nanofibrous scaffold, the method comprising:
forming an electrospinning mixture comprising collagen and antithrombotic glycan; and
electrospinning the electrospinning mixture to form a synthetic nanofibrous scaffold.

14. The method of claim 13, wherein forming an electrospinning mixture comprising collagen and antithrombotic glycan comprises:
forming a first component, wherein forming a first component comprises mixing collagen with a first solvent;
forming a second component, wherein forming a second component comprises mixing the antithrombotic glycan in an aqueous second solvent; and
mixing the first component with the second component to form an electrospinning mixture.

15. The method of claim 13, wherein the electrospin mixture comprises the collagen and the antithrombotic glycan in a mass ratio of about 90:10, respectively, to about 99 to 1, respectively.

16. The method of claim 13, wherein forming the electrospinning mixture occurs prior to electrospinning the electrospinning mixture.

17. The method of claim 13, wherein forming the electrospinning mixture occurs while electrospinning the electrospinning mixture

18. The method of claim 13, wherein forming the electrospinning mixture comprises adding an active agent to the electrospinning mixture.

19. The method of claim 13, further comprising coating an active agent onto the synthetic scaffold.

20. The method of claim 18, wherein the active agent is a therapeutic agent or a biological material, selected from the group consisting of cells, proteins, peptides, nucleic acids, nucleic acid analogs, nucleotides or oligonucleotides, peptide nucleic acids, aptamers, antibodies or fragments or portions thereof, antigens or epitopes, hormones, hormone antagonists, growth factors or recombinant growth factors and fragments and variants thereof, cell attachment mediators, cytokines, enzymes, antibiotics or antimicrobial compounds, viruses, toxins, prodrugs, chemotherapeutic agents, small molecules, drugs, and combinations thereof.
